SEQUENCE LISTING (7 sequences)

SEQ ID NO: 1 - protein, 197 aa, Artificial Sequence
  recombinant human growth hormone including a glycosylation site
MAHHHHFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLENGSPRTGQIFKQTYSKFDTNSHNDDALLKNYGLLYCFRKDMDKVETFLRIVQCRSVEGSCGF

SEQ ID NO: 2 - DNA, 34 bp, Artificial Sequence
  DNA primer
atggggaggctggaaaatggcagcccccggactg

SEQ ID NO: 3 - DNA, 34 bp, Artificial Sequence
  DNA primer
cagtccgggggctgccattttccagcctccccat

SEQ ID NO: 4 - protein, 8 aa, Artificial Sequence
  myc-DDK
DYKDDDDK

SEQ ID NO: 5 - DNA, 39 bp, Artificial Sequence
  primer for RNASE4
aacattcgtagtatctgcaacaccaccaatatccaatgc

SEQ ID NO: 6 - DNA, 39 bp, Artificial Sequence
  primer for RNASE4
gcattggatattggtggtgttgcagatactacgaatgtt

SEQ ID NO: 7 - DNA, 52 bp, Artificial Sequence
  start codon and n-linked glycan sequence
gaggagannngccaccatgaacaucacgaacaucacgaacaucacgggtacc

(19) United States
(12) Patent Application Publication
(10) Pub. No.: US 2011/0105735 A1
(43) Pub. Date: May 5, 2011

(54) METHODS OF PRODUCING AND PURIFYING PROTEINS

(76) Inventors: Heather Desaire, Lawrence, KS (US); Jennifer Laurence, Lawrence, KS (US); Melinda Toumi, Lawrence, KS (US); Jamie Wenke, Nashville, TN (US)

(21) Appl. No.: 12/915,734

(22) Filed: Oct. 29, 2010

Related U.S. Application Data
(60) Provisional application No. 61/256,034, filed on Oct. 29, 2009.

(51) Int. Cl. C07K 14/61; C12P 21/00; C07H 21/04; C12N 15/00; C07K 1/22

(52) U.S. Cl. 530/397; 435/68.1; 536/23.1; 435/455; 530/395

(57) ABSTRACT

Methods of producing and purifying proteins that comprise a glycan tag are provided. In some embodiments, a protein may be modified to include a glycan tag so as to facilitate production of the protein, e.g., by promoting protein secretion and/or promoting protein solubility. In some embodiments, the present disclosure provides methods wherein a protein may be modified to include a glycan tag, which may then be used as an affinity tag for purification.

### CROSS REFERENCE TO RELATED APPLICATIONS

This application claims priority to U.S. Provisional Application No. 61/256,034, filed Oct. 29, 2009, which is incorporated herein by reference.

### STATEMENT REGARDING FEDERALLY SPONSORED RESEARCH OR DEVELOPMENT

This disclosure was made with support under Grant Number P20 RR016475, awarded by National Center for Research Resources, a component of National Institutes of Health and under Grant Number 0645120 awarded by National Science Foundation. The U.S. government has certain rights in the invention.

### BACKGROUND

Recombinant protein production is pivotal in multiple industries, e.g. pharmaceuticals, biomedical diagnostics, and life sciences research, with biopharmaceuticals alone comprising a $40 billion market. In any setting, producing adequate quantities of highly purified protein is a formidable challenge. One component in meeting this challenge is deciding upon an appropriate protein expression system. Typically, a cellular platform such as Escherichia coli (E. coli) is the first choice. Unfortunately, the expression of high levels of recombinant proteins in E. coli often result in the formation of inactive, denatured proteins that accumulate in intracellular aggregates known as insoluble inclusion bodies, which must then be dismantled using a series of trial and error experiments designed to extract, solubilize, and refold the protein. Whenever the protein is expressed in a soluble and folded form, the protein is contained within a complex biological matrix and extensive manipulations are then required to achieve sufficient purity.

In addition to the expression system, selecting an appropriate purification method is also critical in meeting the protein production challenge. Recombinantly expressed proteins are commonly purified using one or more affinity tags, paired with a chromatographic method such as immobilized metal affinity chromatography (IMAC). Affinity tags, which typically consist of amino acid residues selected for their chemical behavior and/or binding epitopes, are engineered into the protein sequence, commonly at the C or N terminus. The most widely used affinity tag is the polyhistidine tag. This repeating sequence of His residues (His-tag®) may also require additional amino acids near the tag location to direct tag removal using an exo- or endopeptidase. Polyhistidine tags have been reported to negatively impact the structure, activity, or binding of some proteins, and they often decrease the amount of protein produced when expressed in eukaryotic culture systems. Other tagging methods, such as biotinylation, may be inappropriate for high throughput protein production due to cost and robustness issues. The development of a new strategy that overcomes commonly encountered problems in protein production has the potential to make significant contributions to science and medicine.

### SUMMARY

The present disclosure is directed to methods of producing and purifying proteins. More specifically, the methods of the present disclosure comprise producing and purifying proteins that comprise a glycosylation site that is not naturally occurring, wherein the glycan at the site may be used as an affinity tag, as a means to increase protein yield/secretion, and/or as a means to solubilize the protein.

The present disclosure is directed to a new protein production platform that may circumvent some of the most challenging complications that arise during protein production and purification, such as extracting inclusion body proteins, generating significant quantities of natively folded protein, and issues with protein solubility. In some embodiments, the methods of the present disclosure comprise adding glycosylation to a protein to provide a unique glycan affinity tag and/or to signal secretion (in eukaryotic cell-based systems) and/or to promote protein solubility. The resulting glycosylated protein may be purified using affinity chromatography or other means. Protein production and glycosylation can be accomplished in cell-based or cell-free systems, and a large variety of glycans may be utilized. After the glycosylated protein is isolated, the glycan may be completely removed or modified, if desired, and an unmodified or modified protein may be generated. In some embodiments, leaving the glycan attached to the protein may synergistically improve other qualities of the protein as well, such as solubility. Thus, the methods of the present disclosure may provide a new route to generate high yields of purified, properly folded, highly soluble proteins.

In one embodiment, the present disclosure provides a method comprising modifying a protein to include a glycan tag so as to facilitate production of the protein.

In another embodiment, the present disclosure provides a method comprising modifying a protein to include at least one modification selected from the group consisting of a glycan tag, a glycosylation unit, and a combination thereof; and then modifying the protein to at least partially remove the at least one modification.

In another embodiment, the present disclosure provides a method comprising modifying a protein to include a glycan tag; and retaining the protein by affinity chromatography.

In yet another embodiment, the present disclosure provides a composition comprising a nucleic acid sequence encoding a start codon; a nucleic acid sequence encoding a glycosylation unit; and a nucleic acid sequence encoding a protein, wherein the nucleic acid sequence encoding in the glycosylation unit is present in a region outside the nucleic acid sequence encoding the protein.

In yet another embodiment, the present disclosure provides a composition comprising a nucleic acid sequence encoding a start codon; a nucleic acid sequence encoding a glycosylation unit; and a nucleic acid sequence encoding a protein, wherein the nucleic acid sequence encoding the glycosylation unit is present inside the nucleic acid sequence encoding the protein and proximate to a terminus of the protein.

The features and advantages of the present invention will be apparent to those skilled in the art. While numerous changes may be made by those skilled in the art, such changes are within the spirit of the invention.

### DRAWINGS

For a more complete understanding of the present disclosure, and the advantages thereof, reference is now made to the following descriptions taken in conjunction with the accompanying drawings, in which:

FIG. 1 depicts certain useful nucleotide sequences of the present disclosure, according to certain embodiments, where (A) represents a start codon, (B) represents code for a glycosylation unit, (C) represents a protein of interest, and (D) represents some additional fusion peptide or protein.

FIG. 2 depicts a useful nucleotide sequence of the present disclosure, according to certain embodiments, where (A) represents a start codon, (B) represents code for a glycosylation unit, repeated, (R′) restriction site for endonuclease, (C) represents the gene for Cytochrome P460 2D protein to be produced.

FIG. 3 depicts a useful nucleotide sequence of the present disclosure, according to certain embodiments, where (A) represents a start codon, (D) represents a fusion peptide, (C′) represents part of the protein gene, (B) represents code for a glycosylation unit, and (C″) represents a remaining portion of the protein gene. While not explicitly shown, the glycosylation unit could also be located in the middle of a fusion protein or peptide, and code for more than one glycosylation site could be included.

FIG. 4 is the protein sequence of rhGH-D136N, according to one embodiment of the present invention.

FIG. 5A is an image of a gel depicting SDS-PAGE analysis of proteins, according to one embodiment of the present invention.

FIG. 5B is an image of a gel depicting SDS-PAGE analysis of proteins using a silver staining procedure, according to one embodiment of the present invention.

FIG. 6 is a graph depicting far-UV circular dichroism data for rhGH that was produced and purified, according to one embodiment of the present invention.

FIG. 7 is a graph depicting AP-MALDI-MS data of (a) intact RNA substrate with the sequence 5′-GGUAG-3′, (b) nucleotide products from RNA substrate digestion, after reaction with native RNase B, and (c) nucleotide products from RNA substrate digestion, after reaction with modified RNase B. The MS data show that the RNase cleavage reaction was complete for both the native RNase B and modified RNase B. The star symbol indicates sodiated adducts.

FIG. 8 is an image of a gel depicting SDS-PAGE analysis of proteins, according to one embodiment of the present invention.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure is directed to methods of producing and purifying proteins. More specifically, the methods of the present disclosure comprise producing and purifying proteins that comprise one or more glycan tags which provide some temporary or permanent benefit to protein production, and may subsequently be removed, modified, or left intact.

As used herein, the term “protein” also includes peptides and/or any sequence of amino acids joined together through amide bonds. Peptides of the present invention can vary in size, e.g., from two amino acids to hundreds or thousands of amino acids, which alternatively is referred to as a polypeptide. Additionally, unnatural amino acids are also included. Amino acids that are not gene-encoded may also be used in the present invention. Furthermore, amino acids that have been modified to include reactive groups, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-liphosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

Furthermore, as used herein, the term “glycan tag” refers to glycosylation that is added to a protein by modification of the protein or nucleotides that encode for the protein; the glycosylation is to occur at a non-natural site for glycosylation. “Glycosylation unit” refers to a sequence of amino acids that are modified or added in a protein for the purpose of introducing a glycan tag. In some embodiments, a glycosylation unit may be a single amino acid modification, such as in Example 1. In some embodiments, a glycosylation unit may include the consensus sequence for N-linked glycosylation. In some embodiments, a glycosylation unit may contain more than three amino acids, where some amino acids are present to introduce a glycan tag and other amino acids are present for other reasons, such as for example to provide a cleavage site for subsequent glycosylation unit removal. The term “glycan” refers to any saccharide or oligosaccharide, in free form or attached to another molecule that can be hydrolyzed into these units. The term “non-natural” generally means not from nature.

Depending on the desired application, a variety of glycans may be suitable for use in the glycan tags of the present disclosure. Examples of suitable glycosylation types and subtypes may include, but are not limited to, asparagine linked (N-linked), O-linked, C-type, glycation, glycosphingolipidation, glycosaminoglycans, glycogenin, phosphoglycosylation, and a combination thereof. One of ordinary skill in the art with the benefit of this disclosure would be able to determine the appropriate type of modification to obtain the desired glycan tag.

In certain embodiments, the present disclosure provides methods wherein a protein may be modified to include a glycan tag so as to facilitate production of the protein, e.g., by promoting protein secretion and/or promoting protein solubility. In some embodiments, the present disclosure provides methods wherein a protein may be modified to include a glycan tag, which may then be used as an affinity tag for purification. The methods of the present disclosure may provide several benefits. For example, in some embodiments, the addition of a glycan tag of the present disclosure may increase the stability of the protein, which may be particularly advantageous because polyhistidine tags are known in some cases known to negatively impact protein stability. Furthermore, in some embodiments, the addition of a glycan tag of the present disclosure may prevent aggregation of proteins, decrease protein degradation, increase protein yield through an increase in protein secretion (in eukaryotic cell-based systems) and/or increase the solubility of the proteins.

Proteins suitable for use in the methods of the present invention may include any naturally occurring or synthetic protein. In one embodiment, proteins suitable for use in the methods of the present invention may comprise a protein that is not naturally glycosylated. In other embodiments, proteins suitable for use in the methods of the present invention may comprise a naturally glycosylated protein to which one or more glycan tags or glycosylation units may be added to improve the solubility or secretion thereof. In another embodiment, the glycosylation state of the protein may be unknown. Examples of suitable proteins may include, but are not limited to, biopolymers, cytokines, growth factors, binding proteins, enzymes, and the like. In one specific embodiment, the protein may encode human growth hormone (hGH).

According to certain embodiments, a protein may be modified to include a glycan tag by modifying a nucleotide sequence encoding a target protein to include a glycosylation unit via recombinant DNA methodology so that upon protein production, the protein comprises a glycan tag. As used herein, the term “nucleotide sequence” includes a sequence that may be comprised of DNA using the language GCAT or RNA using the language GCAU, or synthetic forms of nucleotides or combinations thereof. The nucleotide sequences may be linear, double stranded, and/or in circular form. Nucleotide sequences are conventionally read in the 5′ to 3′ direction, but certain methods of protein production may use 3′ to 5′ or other orientations. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with gene, cDNA, and mRNA encoded by a gene. The term “gene” means the segment of DNA involved in producing a polypeptide chain. It may include regions preceding and following the coding region (leader and trailer) as well as intervening sequences (introns) between individual coding segments (exons).

In some embodiments, a nucleotide sequence coding for the desired protein containing both the target protein sequence and a glycosylation unit is prepared using methods known to those of skill in the art. These methods may generally involve preparing oligonucleotides coding for fragments of the desired coding sequence and for the complementary sequence thereof. The oligonucleotides are designed to provide overlap of one fragment of the coding sequence with two fragments of the complementary sequence and vice versa. The oligonucleotides are paired and joined, ultimately producing the desired sequence. The sequence may then be inserted into a cloning vector at a location that permits the glycosylated protein to be expressed. The sequence could be produced using any single or combination of synthetic, semi-synthetic or biosynthetic methods.

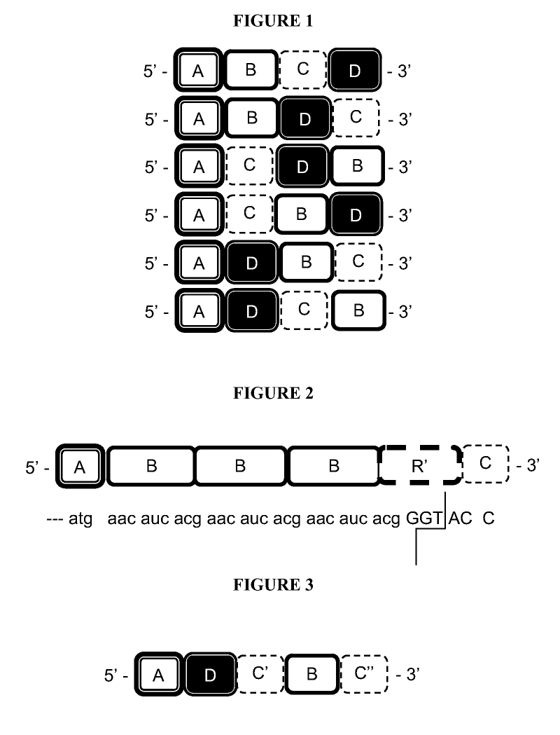

According to certain embodiments, a nucleotide sequence may be constructed to include discrete sections that code for a start codon, a code for one or more glycosylation units, code for the gene for the protein that is targeted for production, and code for an additional fusion protein or peptide. An individual nucleotide sequence may contain code for some of the sections, all of the sections, or a combination that includes a repetition of one or more sections. The nucleotide sequence can be prepared using methods known to those of skill in the art. Preparation methods may make use of known restriction sites to be used with enzymes such as endonucleases, whereas it may be useful to remove, add in, rearrange, or repeat one or more of the sections of the nucleotide sequence. FIG. 1 displays examples of section arrangements that may useful in protein production according to certain embodiments of the present disclosure. In FIG. 3, examples of nucleotide sequence arrangement that comprise putting the glycosylation unit within another section are shown. A glycosylation unit may be positioned anywhere within the sequence of the gene of interest and/or any fusion partner, and in as many positions and/or copies as desired. The strategy depicted in FIG. 3 is the same strategy used in Examples 1 and 3.

In other embodiments, a protein may be modified to include a glycan tag by appending a glycan to the protein as a co/post-translational modification during or after its production, thereby utilizing the glycosylation machinery located within or derived from any expression host (e.g., organelles or their contents). In other embodiments, a glycan tag may be appended to the protein using a glycosylatransferase, such as endo-β-N-acetylglucosaminidase (Endo M). In other embodiments, a glycan tag such as a glycoside may be lengthened and then enzymatically appended to the protein by a chemo-enzymatic method.

Glycan tags suitable for use in the methods of the present invention may be incorporated into a suitable protein at any point, irrespective of the method used to introduce the glycan tag, and thus are not restricted to a protein's termini. One of ordinary skill in the art with the benefit of this disclosure would be able to determine an appropriate location for a glycan tag. In some embodiments, where a glycan tag is introduced into a protein through the addition of a glycosylation unit, the glycosylation unit may be specifically inserted into a loop of the protein, away from an active site, or proximate to either termini of a protein. In certain embodiments, the ability to select the location of a glycosylation unit may be advantageous because certain proteins, such as hGH, may have binding motifs close to or including the terminal parts of a protein.

Protein production and glycosylation can be accomplished in cell-based or cell-free systems. In some embodiments, genetic constructs for the expression of a target protein may be introduced into host cells using well known techniques such as infection, transduction, transfection, electroporation, transformation, and hybridization. When expressing proteins in a cell-based system, the glycan tag may be recognized as a signal for extracellular secretion in eukaryotes, and this modification is thought to not only increase the yield of the protein but also to protect the recombinant protein from aggregating, overcoming a major obstacle often encountered when using prokaryotes such as E. coli as a host.

After a protein has been modified to include a glycan tag, in some embodiments, the glycan tag may then be used as an affinity tag to separate the protein from impurities using affinity chromatography or other means. In some embodiments, lectin affinity chromatography may be used. In preparing a suitable affinity column, one of skill in the art will recognize that the affinity column will need to have specificity for the particular type of glycan tag attached to the protein. One of ordinary skill in the art with the benefit of this disclosure will be able to select an appropriate affinity reagent based on the type of glycosylation on the protein being purified. The glycosylation profile can be predicted, based on the type of cell line used, or it can be characterized by methods available to one with skill in the art. The methods of this disclosure may be combined with any method of separation and/or purification, such as, but not limited to, size exclusion chromatography, other protein fusion pairs, or a combination of affinity tags or general methodologies, such as salting out approaches.

In some embodiments, after the glycosylated protein is isolated, a glycan tag may be modified or completely removed, if desired, and the unmodified protein may be generated. In some embodiments, a glycan tag may be removed or modified by any known method, including but not limited to, enzymatic, non-enzymatic, and/or chemical means. To fully or partially remove a glycan tag from the protein, enzymes including, but not limited to, PNGase F, EndoH, EndoF, or other glycosidases may be used; chemical methods may also be employed.

In some embodiments, after the glycosylated protein is isolated, the glycosylation unit may be removed. Examples of suitable chemical cleavage methods to modify or remove the glycosylation unit of the present disclosure may include those disclosed in “Chemical Cleavage of Proteins in Solution”, Dan L. Crimmins, Sheenah M. Mische, Nancy D. Denslow, Current Protocols in Protein Science, Unit Number 22.4, June 2005 and “Cyanylation of rhodanese by 2-nitro-5-thiocyanobenzoic acid”, Laura Pecci, Carlo Cannella, Bernardo Pensa, Mara Costa and Doriano Cavallini, Biochimica et Biophysica Acta (BBA)—Protein Structure, Volume 623, Issue 2, Jun. 26, 1980, Pages 348-353. Furthermore, the glycosylation unit may be cleaved using enzymatic methods, for example using proteases.

In some embodiments, however, leaving a glycan tag attached to the protein may synergistically improve other qualities of the protein, such as solubility. For example, a glycan tag may be used to increase the solubility of the protein targeted for production. While not wishing to be bound to any particular theory, it is currently believed that because glycans consist of small carbohydrate units bonded together, which are known to be very hydrophilic and thus, highly soluble in aqueous solutions, a glycan tag is expected to improve the solubility of a protein to which it is attached when in an aqueous solution.

### Example 1

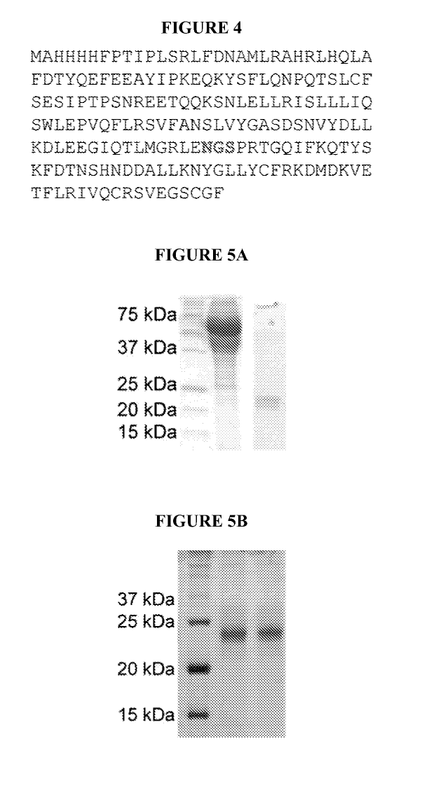

A novel recombinant human growth hormone (rhGH) mutant was designed that incorporated an N-linked glycan. Found endogenously, hGH is a non-glycosylated 22 kDa protein that is critical to proper growth and metabolism. A glycosylation unit was introduced into the rhGH sequence using mutagenesis to change the amino acid sequence from DGS to NGS at residue D136. FIG. 4 shows the verified sequence, with the N-linked site indicated in bold (SEQ ID NO: 1). This particular amino acid sequence is recognized by cellular machinery within eukaryotic cells as a signal to attach, and then modify, an N-linked glycan. This rhGH-D136N mutant was expressed in Chinese hamster ovary cells (CHO-K1), the most commonly used mammalian expression hosts, which are known for producing complex N-linked glycans at the consensus sequence N-X-S/T. Purification was performed using lectin affinity chromatography, and the glycan was removed using an endoglycosidase. The pure, nonglycosylated product was characterized using gel electrophoresis (SDS-PAGE) and circular dichroism (CD) and was found to possess the desired molecular weight and secondary structure.

Materials

The CHO-K1 cells were a gift from Dr. Jeff Krise (University of Kansas, Department of Pharmaceutical Chemistry, Lawrence, Kans.). Water was purified using the Millipore Direct Q-3 system (Millipore, Billerica, Mass.). All materials were obtained from Sigma unless otherwise specified. All solutions were passed through a 0.2 μm filter.

Construction of Recombinant Template Plasmid

To create the rhGH plasmid, the mammalian vector pCMV6-XL5 (Origene Technologies Inc., Rockville, Md.), which encodes ampicillin resistance, was cut with two restriction enzymes XbaI and PstI (Promega, Madison, Wis.). The hGH gene was purchased from Origene Technologies Inc. (SC-3300088) and amplified using PCR with primers encoding these same restriction sites. The 5′ native signal sequence was included in the construct to enable secretion of the protein. The DNA also included a polyhistidine tag consisting of four histidine residues. The amplified product encoding hGH (IDT, Coralville, Iowa) was treated with the same restriction enzymes. Products of both reactions were purified separately utilizing the Wizard® SV gel and PCR clean-up system (Promega, Madison, Wis.). To insert the hGH gene into the chosen vector, T4 DNA Ligase (Promega, Madison, Wis.) was incubated at 15° C. Competent XL-1 Blue E. coli cells (Stratagene) were transformed using the plasmid, plated on Luria broth (LB) agar plates containing 100 μg/mL ampicillin. A colony was selected and used to inoculate 10 mL of selective LB medium. The resultant DNA was purified using a QIAprep Spin Miniprep Kit (QIAGEN, Valencia, Calif.) and quantified using a standard absorption assay on a Nanodrop® ND-1000 Spectrophotometer (Thermo Scientific, Wilmington, Del.). Sequencing was performed by Northwoods DNA, Inc. (Bemidji, Minn.). DNA sequence data were analyzed using the FinchTV application and confirmed to be correct.

Site-Directed Mutagenesis of Glycosylation Unit

The verified plasmid was used as a template for modification to include an N-linked glycan. Primers (Integrated DNA Technologies, Inc., Coralville, Iowa) were designed to generate the rhGH-D136N mutant: (5′ to 3′) atg ggg agg ctg gaa AAT ggc agc ccc cgg act g (leading) (SEQ ID NO: 2) and cag tcc ggg ggc tgc cAT Ttt cca gcc tcc cca t (reverse) (SEQ ID NO: 3), where the mutated bases are indicated using capital letters. Primers were combined with the DNA template, PfuTurbo Polymerase® (Stratagene, La Jolla, Calif.) and other components, according to the manufacturer's directions for the QuikChange mutagenesis procedure.

The mutagenesis product was digested with 1 μL of DpnI enzyme (New England Biosciences, Ipswich, Mass.) to selectively destroy the template DNA. The product was then used to transform competent XL-1 Blue E. coli cells. Transformed cells were plated onto LB agar plates containing ampicillin, grown overnight at 37° C., and a single colony was selected to inoculate liquid LB medium. After overnight growth, the resultant DNA was isolated and purified using the Qiaspin Miniprep Kit (Qiagen, Valencia, Calif.). All miniprep DNA was quantified as described above and shown by DNA sequencing to be the desired product.

Cell Culture and Transfection

CHO-K1 cells were seeded and maintained using complete high glucose Dulbecco's Modified Eagle Medium (DMEM) (Thermo Scientific Hyclone, Logan, Utah). The medium also contained supplemental MEM Non-Essential Amino acids 100× (ATCC, Manassas, Va.), 10% fetal bovine serum (Mediatech, Inc. Manassas, Va.), 100× penicillin/streptomycin (ATCC, Manassas, Va.), and 150 mM L-Proline. Cells were maintained in T-75 flasks (BD, Franklin Lakes, N.J.) with 24 mL of medium and kept at 37° C. and 5% CO₂. Passage was accomplished by using two wash steps with pre-warmed phosphate buffered saline (PBS) alone and PBS containing trypsin to remove the adherent cells from the surface of the flask. The suspension was spun at 3800×g for 2 minutes, the supernatant was removed via vacuum, and the cells were resuspended via gentle pipetting into fresh culture medium as described above.

Cells were allowed to grow to 50% confluency and then transfection was accomplished with 4 μg of rhGH-D136N DNA and 20 μL of Turbofectin 8.0 transfection reagent (OriGene Technologies, Inc., Rockville, Md.) per T-75 flask. The supernatant was decanted after 24 hours and the medium replaced. After an additional 24 hours the supernatant was collected and concentrated to 0.5 mL using centrifugal filtration devices (Millipore, Billerica, Mass.) with a 10 kDa molecular weight cut off (MWCO) at 9000×g.

Lectin Affinity Chromatography (LAC) Purification

A cartridge containing Maackia amurensis leukoagglutinin (MAL) lectin resin (Qiagen, Valencia, Calif.) was equilibrated according to the manufacturer's directions using the indicated binding/wash buffer. The concentrated supernatant (0.5 mL) from a single supernatant collection was mixed with an equal volume of binding/wash buffer and loaded onto the column at 1 mL/minute. The column was washed using 50 mL of binding/wash buffer at 4 mL/min, and the rhGH was eluted into 50 mL of elution buffer containing 200 mM lactose. The elution fraction was concentrated to 0.5 mL as before. The protein content was quantified using a standard Bradford assay. The rhGH protein was dialyzed using Slide-A-Lyzer dialysis cassette with a 10 kDa MWCO (Pierce, Rockford, Ill.) into 10 mM sodium citrate and 150 mM sodium chloride, with 3 (1 L) exchanges.

Immobilized Metal Affinity Chromatography (IMAC) Purification

A prepared 5-mL column containing Chelating resin (GE Healthcare) charged with nickel was equilibrated. A sample of rhGH-D136N, which was purified using LAC and quantified with a standard Bradford assay, was mixed with an equal volume of loading buffer and was loaded onto the column. Loading buffer containing 50 mM TRIS-HCl, 150 mM sodium chloride, was adjusted with sodium hydroxide to pH 8.0 and was filtered (0.2 μm). Rinsing buffer was the same, except it also contained 20 mM imidazole. The elution buffer was the same as the loading buffer, except it also contained 200 mM imidazole. The column was washed using 100 mL of loading buffer; rinsed using 50 mL of rinse buffer, and elution was performed via syringe using 16 mL of elution buffer. The collected fractions were analyzed using SDS-PAGE and their protein content quantified using a standard Bradford assay.

SDS-PAGE and Protein Concentration Determination

12% SDS-PAGE TRIS/glycine gels were generated for electrophoresis. Protein samples were mixed with 2× reducing Laemmli loading buffer and water (1:3:3) and boiled for 15 minutes. The samples were loaded onto the gel, and electrophoresis was performed at 123 V. Precision Plus unstained protein ladder (Bio-Rad, Hercules, Calif.) was utilized in separate lane(s) for molecular weight approximation. Gels were first stained using Coomassie (R-250) and then fully destained (using acetic acid:methanol 2:3) prior to silver staining.

As seen in FIG. 5A, a very large band at approximately 70 kDa is visible along with several other bands, including a distinct band between the 20 kDa and 25 kDa markers. The large band, in the center lane, is the correct molecular weight for bovine serum albumin, which is added to the culture medium. The rhGH band positioned slightly above the 20 kDa marker is more evident after purification using LAC, as observed in the second lane to the right in FIG. 5A. A portion of the purified rhGH sample was subjected to enzymatic deglycosylation and then SDS-PAGE analysis. The band on the right is from an aliquot of the glycosylated protein, rhGH-D136N, and in the center lane is a single band from the deglycosylated product. No significant bands other than the purified rhGH are visible using the silver staining procedure. (FIG. 5B.)

After separate purification using LAC and IMAC, the purified glycosylated rhGH-D136N protein concentration was determined using a standard Bradford assay. The resulting values are displayed below in Table 1.

TABLE 1

Sample

| | LAC | IMAC |
|---|---|---|
| | Purified | Elution |
| | mg/mL | mg/mL |
| Concentration | 1.67 | Not |
| | | detected |

From a single 24-hour collection of the supernatant, 1.67 mg of glycosylated rhGH protein was recovered from 24 mL of medium. The rhGH concentration was determined to be 1.67 mg/mL after protein purification and concentration. To compare the glycan affinity tag method to purification via affinity to the polyhistidine tag, the purified rhGH was loaded onto an IMAC column. The collected fractions were concentrated as described above. No protein was detected in the second wash fraction, the rinse fraction or the elution fraction, as indicated in Table 1. Analysis of the first wash fraction showed the rhGH-D136N protein concentration to be 1.27 mg/mL, after concentration to approximately 1 mL. This indicates that the metal affinity method was completely ineffective at binding the polyhistadine tagged protein, since the protein should have bound to the column and only eluted during the elution step.

Purification Tag Removal

Purified, glycosylated rhGH was treated with N-Glycosidase F (PNGase F) from Elizabethkingia meningosepticum (CalBioChem, San Diego, Calif.) to remove the glycan and produce rhGH with the original sequence intact by adding 1 μL of 500 units/mL to approximately 2 mg of protein, and incubated for 24 hours at 37° C. An aliquot of rhGH protein was loaded onto the MAL lectin cartridge using the above procedure. The collected fractions were analyzed using both SDS-PAGE and a Bradford assay to determine the final amount of purified, nonglycosylated rhGH. The band in the center lane of the SDS-PAGE gel in FIG. 5A is the deglycosylated, purified protein.

CD Secondary Structure Estimation

The purified protein was diluted to 0.5 mg/mL in 10 mM sodium citrate and 150 mM sodium chloride for structural analysis and placed in a jacketed, quartz, 1.0-mm pathlength sample cell. Far UV CD measurements were made using a Jasco J-715 spectropolarimeter (Tokyo, Japan) with an attached circulating water bath that was built in-house. Experiments were performed at 10° C., with a sensitivity of 100 mdeg and scan speed of 10 nm/min. The experiments were conducted under constant nitrogen flow. Scans were performed from 260 to 185 nm. Multiple spectra were averaged, smoothed and baseline corrected by subtracting signal from background solutions prior to analysis. The publicly available DICHROWEB server was used, with the CDSSTR algorithm, to calculate the percent protein secondary structure.

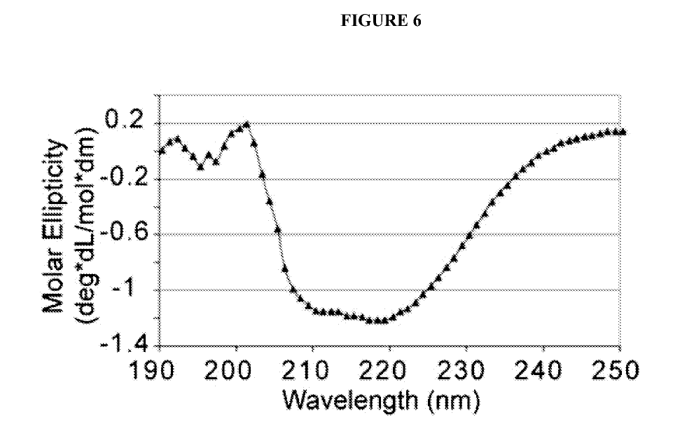

Secondary structure of the deglycosylated protein, containing the native hGH sequence, was characterized using far-UV circular dichroism. The spectrum is shown in FIG. 6. The far-UV scan of the rhGH protein was compared to measured values reported in the literature. Minima in the spectrum are observed at wavelengths 209 nm and also at approximately 222 nm. The spectrum indicates that a large portion of the secondary structure present in the protein sample is alpha helical in character. Endogenous hGH is known to have alpha helices account for 50-60% of secondary structural characteristics. To quantify the helical content of the rhGH sample, the CD data was analyzed using the publicly available, web-based DICHROWEB program.

The secondary structure calculation results were returned from the server as percent secondary structure and are displayed in Table 2.

TABLE 2

| rhGH | Helix | Strand | Turn | Unordered | Total |
|---|---|---|---|---|---|
| D136N mutant | 60% | 15% | 7% | 19% | 101% |

The alpha-helical character was calculated to be 60% for the deglycosylated rhGH protein, which is within the range of acceptable values found in the literature. These data indicate adequate quantities of protein have been produced with the intended secondary structure intact and thus support the methods of the present disclosure.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Example 2

RNase B, a glycoprotein, was used to demonstrate that a glycan present on a protein may be extracellularly modified and the functionality of the glycoprotein may still be maintained. RNase B is a 15 kDa enzyme that contains that contains a single N-linked glycosylation unit at N³⁴, and consists of both α-helices and β-strands in its structure.

Materials

HPLC grade methanol was obtained from Thermo Fisher Scientific (Fairlawn, N.J.), RNA substrate was obtained from Integrated DNA Technologies (Coralville, Iowa), and sodium bicarbonate was purchased from Fluka (Milwaukee, Wis.). Water was purified using the Millipore Direct Q-3 system (Millipore, Billerica, Mass.), and all other reagents were purchased from Sigma-Aldrich (St. Louis, Mo.).

Glycan Modification

Approximately 500 μg of bovine pancreatic RNase B was added to 300 μL of 10 mM NH₄HCO₃ buffer, and the solution was adjusted to pH 5.0 using HCl. A commercially available α-mannosidase was added in an enzyme:glycoprotein ratio of 1:1000 (mol/mol), and incubated for 72 hours at 37° C. A second, identical sample of RNase B was treated to the same conditions, except the mannosidase was not added.

Mass Spectrometry

A portion (150 μL) of the glycoprotein samples was subjected to DTT in excess for 1 hour at 60° C., then diluted to 5 μM using 80:20 MeOH:H₂O, and 0.5% acetic acid. MS data were collected using a Thermo Fisher Scientific (San Jose, Calif.) hybrid linear ion-trap Fourier transform ion cyclotron resonance mass spectrometer (LTQ-FT-ICR-MS). Direct infusion was performed at 2 μL/min for electrospray ionization in the positive ion mode. The spray voltage was set at 2.8 kV, and the N₂ sheath gas was set to 10.0 psi. Ion desolvation was aided by heating the ion transfer tube to a temperature of 250° C. Resolution was set to 100,000 for the ion m/z 400. All parameters were adjusted using the instrument software Excalibur, version 2.0.

Circular Dichroism

Far UV CD measurements were made using a Jasco J-715 spectropolarimeter (Tokyo, Japan) with an attached circulating water bath. Portions of the native and the modified glycoproteins were diluted with water to a final concentration of 71 μM, and 250 μL of sample was placed in a jacketed, quartz, 1.0 mm pathlength sample cell. Experiments were performed at 10° C., with a sensitivity of 100 mdeg and scan speed of 20 nm/min. The experiments were conducted under constant nitrogen flow. Scans were performed from 260 to 190 nm in triplicate. All spectra were smoothed and baseline corrected by subtracting signal from background solutions prior to analysis. The publicly available DICHROWEB server was used, with the SELCON3 algorithm, to estimate the percent glycoprotein secondary structure for α-helices, β-strands, β-turns, and disordered structural components.

Activity Assay

RNA substrate, 5′-GGUAG-3′, was diluted to 0.1 mg/mL with RNase free water. A small amount of the RNA substrate was mixed with native RNase B, in an enzyme:substrate ratio of 1:10,000 (mol/mol) and allowed to react for 5 minutes. The reaction was quenched using 0.1% (v/v) diethyl pyrocarbonate. One microliter of product was combined with 1 mL of MALDI matrix (15 mg/mL dihydroxybenzoic acid in 80:20 acetonitrile:water), spotted, and allowed to dry at 60° C. for 30 minutes. The reaction was repeated using the modified RNase B sample. Mass spectral data were collected in negative ion mode using a quadrupole ion trap mass spectrometer (LCQ Advantage; Thermo Fisher Scientific) and an atmospheric pressure matrix-assisted laser desorption ionization (AP-MALDI) source (MassTech, Columbia, Md.). The ion transfer tube was heated to 250° C. to assist with desolvation of ions. The nitrogen laser was operated at 337 nm, with a 4 ns pulse width, and controlled using AP Maldi Target software, version 3.4. The voltage applied to the MALDI plate was set to 3.0 kV. A total of 333 laser shots were averaged into one spectrum prior to data analysis. Data analysis was performed using the instrument software Excalibur, version 1.3.

Results

Enzymatic Glycan Modification and Verification by MS

RNase B has five naturally occurring glycoforms, ranging from 5 to 9 mannose residues (man₅-man₉), at its single N-linked glycosylation unit. RNase B was reacted with α-mannosidase to trim the glycans.

Glycan compositional data for the glycoprotein was acquired before and after glycan trimming. The model glycoprotein could be characterized by direct analysis using MS techniques. This was accomplished by analyzing the whole glycoprotein with electrospray ionization in the positive ion mode, using an FTICR-MS. RNase B contains four disulfide bonds, and if reduced, charge states are observed well within the required m/z range. Reduction of disulfide bonds was performed with DTT in excess. Upon analysis of the reduced native and modified glycoproteins, a distribution of charge states, +8 through +11, were observed. The five naturally occurring glycoforms of the native glycoprotein were observed in all charge states detected, with man₅ accounting for the largest peak for the native glycoprotein.

The glycoforms observed in the modified RNase B sample were expected to differ from the native protein by possessing fewer mannose residues, due to the reaction with the exoglycosidase, α-mannosidase. This glycoenzyme was expected to sequentially trim terminal mannose residues from the glycan structures of RNase B, based on characterization of its specific action. Mass spectrometry experiments were repeated using the modified RNase B sample. In the +9 charge state, three glycoforms of the modified glycoprotein are detected: man₂ at m/z 1602.45, man₃ at m/z 1620.57, and man₄ at m/z 1638.57.

Structural Measurements

The data below describes the monitoring of the glycoprotein for changes in protein structure. CD spectroscopic analysis was performed on the native and modified glycoprotein samples in the far UV wavelength region (260-190 nm) to monitor for differences in secondary structure. Although both samples produced broad signals, an indication of an αβ protein, a slight increase in signal over the α-helical wavelength region (˜209 nm) was present in the modified glycoprotein sample. The results were analyzed using the web-based DICHROWEB server, and the percent secondary structure estimation results are presented in Table 3 below as calculated by the server.

TABLE 3

| RNase B | Helix | Strand | Turns | Unordered | Total % |
|---|---|---|---|---|---|
| Native | 22 ± 1% | 27 ± 1% | 21 ± 1% | 31 ± 1% | 101 |
| Modified | 31 ± 8% | 23 ± 4% | 18 ± 2% | 27 ± 3% | 99 |

Native RNase B was estimated to have α-helices accounting for 22±1% of its overall structure, and 27±1% of its structure was attributed to β-strands. The modified RNase B was characterized as containing 31±8% α-helical and 23±4% β-stranded structures. The rest of the protein was estimated to contain turns and disordered structure. For each of the features identified for this protein, the percent contributions are similar for the native and modified structure, and the absolute differences in the values are generally within the margin of error of the measurement. These data demonstrate that the modified protein had similar secondary structure to the native protein.

RNase B Functional Assay

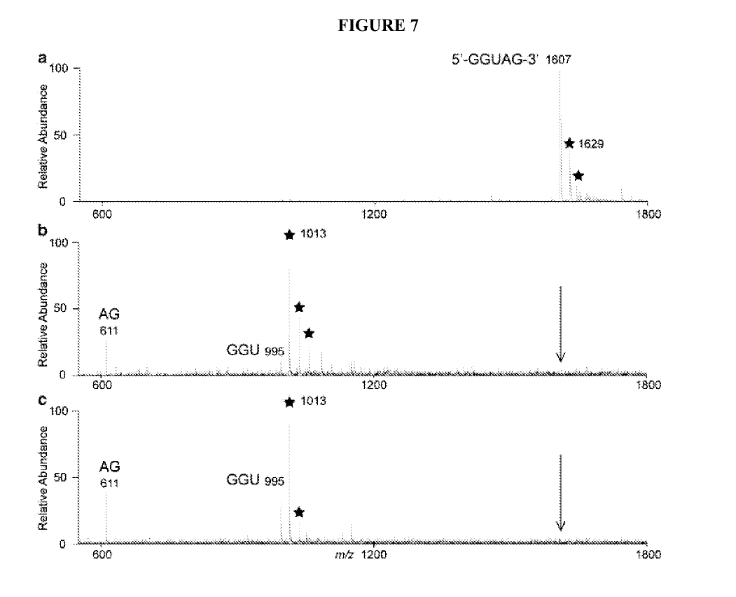

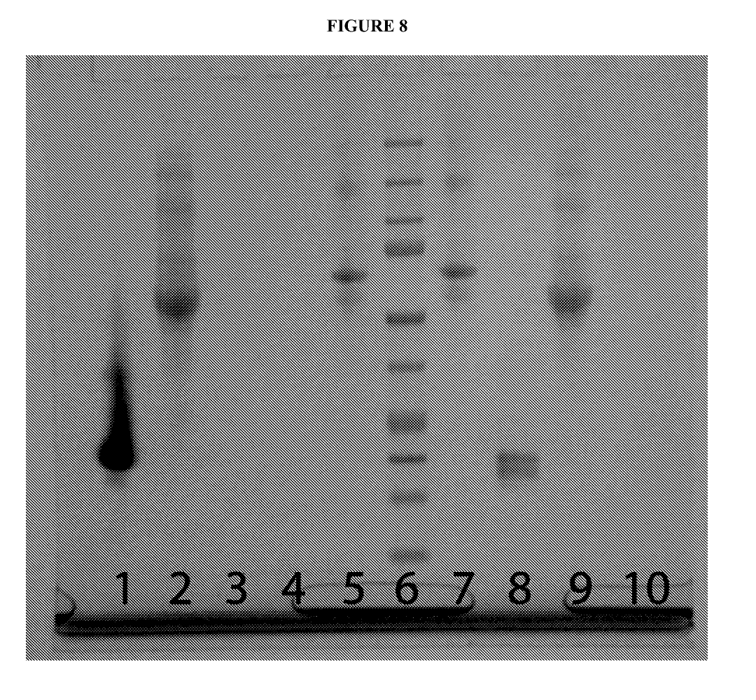

The model glycoprotein chosen, RNase B, is known to catalyze the cleavage of RNA substrates via hydrolysis of the 3′,5′-phosphodiester bonds. The modified RNase B sample's ability to catalyze RNA depolymerization was not expected to be negatively affected by enzymatic glycan trimming, as the removal of mannose residues is thought to promote RNase B activity. In addition, the nonglycosylated analog to RNase B, RNase A, is known to also perform similar functions. To demonstrate retention of activity after extracellular glycan modification, an RNA substrate with the sequence 5′-GGUAG-3′ was reacted with the native glycoprotein, and then the experiment was repeated with the modified glycoprotein. The products produced from cleavage of the RNA substrate were detected using AP-MALDI, coupled to a quadrupole ion trap mass spectrometer. In FIG. 7, AP-MALDI-MS data acquired in the negative ion mode are shown for the intact RNA substrate (Panel a), along with the RNA substrate after reacting with the native RNase B (Panel b). Panel c shows the RNA after reacting with the RNase B that has undergone enzymatic glycan trimming. The base peak in Panel a, at m/z 1607, represents the intact substrate with sequence 5′-GGUAG-3′, and its sodiated adducts, for example, at m/z 1629, are labeled with a star symbol. In Panel b, the products of the native RNase B reaction are illustrated. The peaks at m/z 611 and m/z 995 indicate that the RNA substrate was cleaved between the uridine and adenosine substituents, leaving the two nucleotides with base compositions AG and GGU, respectively. No intact substrate was detected in either experiment as indicated by the arrows in Panel b and c. The data in Panel c matched the data in Panel b, thus demonstrating that the native and the modified glycoproteins were both effective at cleaving this particular RNA substrate, under the conditions tested. These results indicate that the glycoprotein RNase B can undergo the extracellular enzymatic glycan trimming process, while retaining its desired function.

### Example 3

A glycan tag was introduced into human ribonuclease 4, variant 3, (RNASE4), a protein which is not naturally glycosylated, to improve protein production. The protein sequence was mutated to possess an asparagine residue in place of serine, at residue 86. This mutation inserted the N-linked glycosylation consensus sequence NTT into the protein's sequence at residue N-86. RNASE4 was also produced without a glycan tag, but instead was expressed with a fusion peptide consisting of eight amino acids at the N terminus. The octapeptide is referred to as the FLAG® tag or as myc-DDK, having the amino acid sequence DYKDDDDK (SEQ ID NO: 4). A commercially available purification method (involving a FLAG affinity column) was used to isolate the RNASE4 with FLAG tag, while the protein with glycosylation appended was isolated using a lectin column. The results were analyzed by gel electrophoresis (SDS-PAGE), to observe a direct comparison of the protein yields for the two expressed proteins, and quantitative analysis was also performed using a Bradford Assay.

Recombinant Plasmids

The RNASE4 gene was purchased in the mammalian vector pCMV6-XL4 from Origene Technologies Inc., (SC107718) and used as a template for site directed mutagenesis to include a glycosylation unit. Site directed mutagenesis was performed using all the same methods as in Example 1, with the only exception being the primers used. The primers for RNASE4 (Integrated DNA Technologies, Inc., Coralville, Iowa) were (5′-3′) aac att cgt agt atc tgc aAc acc acc aat atc caa tgc (leading) (SEQ ID NO: 5) and g cat tgg ata ttg gtg gtg Ttg cag ata cta cga atg tt (reverse) (SEQ ID NO: 6), where the capital letters indicate the mutated bases. The DNA sequencing results (Northwoods DNA Inc., Bemidji, Minn.) indicated that the RNASE4-S86N mutant gene had the correct sequence. The RNASE4-mycDDK plasmid was purchased (Origene, RC214405) and used for transfection directly.

Cell Culture and Transfection

Both RNASE4 vector plasmids were transfected into CHO-K1 cells using the same materials and methods as in Example 1, with the following exceptions. The DMEM cell culture medium contained 5% fetal bovine serum instead of 10%. The flasks used were T-25 instead of T-75, which contained 6 mL of medium instead of 24 mL. Transfection was performed using 4 μg of DNA and 12 μl, of Turbofectin 8.0 transfection reagent (Origene Technologies Inc.) The supernatant of each flask was decanted after 24 hours and the medium replaced. After an additional 24 hours, the supernatant was collected for affinity purification.

Protein Purification Using Lectin Affinity Chromatography

The RNASE4-S86N was collected, filtered using a 0.2 μm filter, and concentrated using a MWCO filter as in Example 1. Briefly, the MWCO filters used in these experiments (Millipore, Billerica, Mass.) were for proteins greater than 10 kDa, and were centrifuged at 9000×g. In separate control experiments, it was verified that RNASE does not pass through MWCO filters of this cut-off. Lectin affinity chromatography was performed in the same manner as Example 1, except the final elution step was concentrated to a final volume of 400 μL using a MWCO filter. No dialysis was performed.

Protein Purification Using the mycDDK Tag

The supernatant of the RNASE4-mycDDK was used for the purification method described below. The FLAG® M Purification Kit (Sigma, St. Louis, Mo.) was used according to the manufacturer's instructions. All steps were performed at 4° C. The resin was prepared as instructed, the medium supernatant was added to the resin, and the batch method was used to promote antibody adsorption to the tagged RNASE4, with an incubation time of 3 hours. The resin was collected in the column provided in the kit, and rinsed with Wash Buffer. Samples of eluting Wash Buffer were collected from the column directly into a quartz cuvette, and absorbance measurements were obtained at 280 nm, where aromatic amino acids absorb UV light. The column was rinsed with Wash Buffer until the measurements (A₂₈₀) were <0.05, and then the elution step was performed using the 3×FLAG peptide, in a total volume of 5 mL. The elution fraction was then concentrated to a final volume of 0.4 mL using MWCO filters as described above.

SDS-PAGE Analysis

SDS-PAGE 4-12% Bis-TRIS Midi gels (Invitrogen, Carlsbad, Calif.) were used for electrophoretic analysis. A 25 μl, aliquot of each RNASE4 protein sample was mixed with 75 μL of 2× reducing Laemmli loading buffer and 75 μL of water, and heated for 10 minutes at 90° C. The samples were loaded using 8 μL volumes, and electrophoresis was performed in MES buffer (Invitrogen) per manufacturer's directions. Precision Plus Protein Dual Color stained protein ladder (Bio-Rad, Hercules, Calif.) was utilized in a separate lane for molecular weight approximation. The gel was stained overnight using Coomassie (R-250). Destaining was accomplished with 20% acetic acid in 50/50 methanol/H₂O until background was clear.

Results

Protein Concentration Determination

The protein concentration for the elution step of each RNASE4 sample was determined using a standard Bradford assay for both samples. The results are listed below in Table 4.

TABLE 4

Sample

| | RNASE4-S86N | RNASE4-mycDDK |
|---|---|---|
| Concentration (mg/mL) | 1.2 | None detected |

The elution fraction from lectin affinity chromatography containing the glycosylated form of RNASE (RNASE4-S86N) was found to have a final concentration of 1.2 mg/mL. The sample had been concentrated to a final volume of 0.4 mL. Thus, 0.48 mg of RNASE4-S86N was obtained from one 24 hour collection of media (the supernatant) from one T-25 flask (initially containing 6 mL of media). The standard Bradford assay did not detect protein in the RNASE4-mycDDK elution fraction sample.

SDS-PAGE Analysis

SDS-PAGE was performed, and the results are shown in FIG. 8. In Lane 1, a sample of commercially available RNase B (Sigma) was used as a reference standard. Lane 2 contains an aliquot of the RNASE4-S86N, prior to protein purification, and the fetal bovine serum proteins in the medium are visible, most notably the bovine albumin near 70 kDa. Lane 3 contains the elution solution from the protein purified with FLAG® affinity chromatography for the RNASE4-mycDDK sample; no bands are observed. Lane 4 contains an aliquot of the RNASE4-S86N flow through collected while using MWCO filters to concentrate the eluent from lectin affinity chromatography. No bands are observed, indicating protein was not lost through this route. Lane 5 contains an aliquot of solution from the wash step, collected during FLAG® purification of the RNASE4-mycDDK protein, and some bands are visible from the fetal bovine serum, as expected. Lane 6 is the protein ladder used to estimate molecular weight. Lane 8 contains RNASE4-S86N from the lectin affinity elution step, and a broad band is visible between the 15 and 20 kDa protein markers, the expected mass of the protein. Lane 9 has an aliquot from the corresponding wash fraction, where fetal bovine serum is detected, as expected. Lanes 7 and 10 are not relevant to this experiment. These results indicate that very little RNASE4-mycDDK protein was expressed, very little protein was purified, or both of these events occurred. The protein loaded on the gel is below the limit of detection (LOD) for Coomassie stain.

### Example 4

A prophetic example is described herein that starts with a DNA construct comprised of special sections that may permit protein production using certain embodiments of the present disclosure. The protein targeted for production in this example is cytochrome p450, a key enzyme in human drug metabolism. However, any other protein could be produced in this manner. The DNA construct is a plasmid, or circular piece of double stranded DNA, which consists of different parts, including a RBS (Ribosomal binding site), followed by the known eukaryotic start codon AUG. The start codon is followed by nucleotides that code for amino acids that generate the N-linked glycan consensus sequence: asparagine (N), any amino acid other than proline, and then serine (S) or threonine (T), or also, possibly, cysteine (C). The nucleotides that code for the glycosylation unit are repeated.

The nucleotide sequence in this example is 5′-g agg aga XXX gcc acc atg aac auc acg aac auc acg aac auc acg GGT ACC-3′ (SEQ ID NO: 7) where X is a variable nucleotide, the underlined nucleotides indicate the start codon, followed by bold nucleotides indicating a nine nucleotide sequence that will code for the amino acids that form the N-linked glycan consensus sequence. These nine nucleotides, which represent one possible N-linked glycan signal are present in this example construct three times. The nucleotide sequence in CAPITAL letters indicates a restriction site that could be used with Kpn I, an endonuclease from Klebsiella pneumoniae. The nucleotide construct is shown in FIG. 2. The gene that codes for cytochrome p450 production, CYP2D6, would be amplified using PCR with appropriate primers, which are designed to incorporate Kpn I restriction sites in the gene. In this example, the gene and the DNA construct would then be digested in a parallel fashion using 1 μg of DNA starting material incubated with 300 units of Kpn I at 37° C. The use of Kpn I would produce sticky and complementary ends on the DNA construct and the protein gene such that the two can be ligated together using 2 μM T4 DNA ligase. The newly formed DNA could be used to transform competent E. coli XL1-Blue cells using a standard heat shock protocol. After growing in NZY⁺ broth for one hour, the transformed E. coli could be plated using agar that contains antibiotic correlating to the antibiotic resistant gene in the DNA plasmid. In this case, ampicillin. The plates could be incubated at 37° C. A single colony could be chosen to inoculate LB broth, which is then incubated on a shaker overnight at 37° C. and 250 RPM. This DNA construct could be purified and used for transformation or transfection in the protein expression host.

Nucleotide sequences, such as the DNA construct used in this example, could be comprised of numerous different sections. The DNA constructs could be used to produce protein containing the glycan tag. In FIGS. 1-3, useful compositions for additional experiments are shown.

In essence, the potential compositions that are envisioned include the following: All permutations shown in FIGS. 1-3; all possible permutations on FIG. 3, where either the protein of interest or the fusion protein/peptide appears partially before the code for the glycosylation unit and partially after the code for the glycosylation unit; all possible permutations involving addition of nucleotide code for more than one glycosylation unit, either adjacent to each other, as shown in FIG. 2, or not adjacent to each other, such as a construct containing: ABD′BD′C′BC′ where A is the start codon, B is code for the glycosylation unit, D′ is code for part of the fusion peptide; C′ is code for part of the protein of interest. Also, it should be noted that the nucleotide sequence that codes for a glycosylation site could be any sequence that codes for the amino acid, Asn, followed by a sequence that codes for any amino acid except proline, followed by a sequence that codes for any of the three amino acids, Ser, Thr, or Cys.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

1. Carlson, B. Biosimilar market fails to meet projections. Genetic Engineering & Biotechnology News 2009, 29, 8.
2. Hunt, I. From gene to protein: a review of new and enabling technologies for multi-parallel protein expression. Protein Expr. Purif. 2005, 40, 1-22.
3. Makrides, S. C. Strategies for achieving high-level expression of genes in Escherichia coli. Microbiol. Rev. 1996, 60, 512-538.
4. Bajorunaite, E.; Sereikaite, J.; Bumelis, V.-A. L-Arginine suppresses aggregation of recombinant growth hormones in refolding process from E. coli inclusion bodies. Protein J. 2007, 26, 547-555.
5. Fujii, T.; Ohkuri, T.; Onodera, R.; Ueda, T. Stable supply of large amounts of human Fab from the inclusion bodies in E. coli. J. Biochem. 2007, 141, 699-707.
6. Chayen, N. E. Turning protein crystallisation from an art into a science. Curr. Opin. Struct. Biol. 2004, 14, 577-583.
7. Arnau, J.; Lauritzen, C.; Petersen, G. E.; Pedersen, J. Current strategies for the use of affinity tags and tag removal for the purification of recombinant proteins. Protein Expr. Purif 2006, 48, 1-13.
8. Waugh, D. S. Making the most of affinity tags. Trends Biotechnol. 2005, 23, 316-320.
9. Terpe, K. Overview of tag protein fusions: from molecular and biochemical fundamentals to commercial systems. Appl. Microbiol. Biotechnol. 2003, 60, 523-533.
10. Chant, A.; Kraemer-Pecore, C. M.; Watkin, R.; Kneale, G. G. Attachment of a histidine tag to the minimal zinc finger protein of the Aspergillus nidulans gene regulatory protein AreA causes a conformational change at the DNA-binding site. Protein Expr. Purif. 2005, 39, 152-159.
11. Freydank, A.-C.; Brandt, W.; Drager, B. Protein structure modeling indicates hexahistidine-tag interference with enzyme activity. Proteins 2008, 72, 173-183.
12. Goel, A.; Colcher, D.; Koo, J.-S.; Booth, B. J. M.; Pavlinkova, G.; Batra, S. K. Relative position of the hexahistidine tag effects binding properties of a tumor-associated single-chain Fv construct. Biochim. Biophys. Acta 2000, 1523, 13-20.
13. Patrick, G. L. An Introduction to Medicinal Chemistry. 2^{nd} Ed. Oxford University Press: New York, 2001, pages 1-122.
14. Laron, Z. Short stature due to genetic defects affecting growth hormone activity. N. Engl. J. Med. 1996, 334, 463-466.
15. Takahashi, Y.; Kaji, H.; Okimura, Y.; Goji, K.; Abe, H.; and Chihara, K. Short stature caused by a mutant growth hormone. N. Engl. J. Med. 1996, 334, 432-436.
16. Salomon, F.; Cuneo, R. C.; Hesp, R.; Sonksen, P. H. The effects of treatment with recombinant human growth hormone on body composition and metabolism in adults with growth hormone deficiency. N. Engl. J. Med. 1989, 321, 1797-1803.
17. Hossler, P.; Khattak, S. F.; Li, Z. J. Optimal and consistent protein glycosylation in mammalian cell culture. Glycobiology 2009, 19, 936-949.
18. Kao, F.-T., Puck, T. T. Genetics of somatic mammalian cells. IV. Properties of Chinese hamster cell mutants with respect to the requirement for proline. Genetics, 1967, 55, 513-524.
19. Devasahayam, M. Factors affecting the expression of recombinant glycoproteins. Indian J. Med. Res. 2007, 126, 22-27.
20. Helenius, A.; Aebi, M. Intracellular functions of N-linked glycans. Science 2001, 291, 2364-2369.
21. Barn, N. B.; Cleland, J. L.; Randolph, T. W. Molten globule intermediate of recombinant human growth hormone: Stabilization with surfactants. Biotechnol. Prog. 1996, 12, 801-809.
22. Wells, J. A.; de Vos, A. M. Structure and function of human growth hormone: Implications for the hematopoietins. Annu. Rev. Biophys. Biomol. Struct. 1993, 22, 329-351.
23. Abdel-Meguid, S. S.; Shieh, H.-S.; Smith, W. W.; Dayringer, H. E.; Violand, B. N.; Bentle, L. A. Three-dimensional structure of a genetically engineered variant of porcine growth hormone. Proc. Natl. Acad. Sci. USA 1987, 84, 6434-6437.
24. de Vos, A. M.; Ultsch, M.; Kossiakoff, A. A. Human growth hormone and extracellular domain of its receptor: Crystal structure of the complex. Science 1992, 255, 306-312.
25. Sreerama, N.; Woody, R. W. A self-consistant method for the analysis of protein secondary structure from circular dichroism. Anal. Biochem. 1993, 209, 32-44.
26. Lobley, A.; Whitmore, L.; Wallace, B. A. DICHROWEB: An interactive website for the analysis of protein secondary structure from circular dichroism spectra. Bioinformatics 2003, 18, 211-212.
27. Lees, J. G.; Miles, A. J.; Wien, F.; Wallace, B. A. A reference database for circular dichroism spectroscopy covering fold and secondary structure space. Bioinformatics 2006, 22, 1955-1962.
28. Whitmore, L.; Wallace, B. A. DICHROWEB, an online server for protein secondary structure analyses from circular dichroism spectroscopic data. Nucleic Acids Res 2004, 32, W668-W673.
29. Sola, R. J.; Griebenow, K. Effects of glycosylation on the stability of protein pharmaceuticals. J. Pharm. Sci. 2009, 98, 1223-1245.
30. Gut, A.; Kappeler, F.; Hyka, N.; Balda, M. S.; Hauri, H.-P.; Matter, K. Carbohydrate-mediated Golgi to cell surface transport and apical targeting of membrane proteins. EMBO J. 1998, 17, 1919-1929.
31. Plummer, T. H. Jr.; Elder, J. H.; Alexander, S.; Phelan, A. W.; Tarentino, A. L. Demonstration of peptide:N-glycosidase F activity in endo-β-N-acetylglucosaminidase F preparations. J. Biol. Chem. 1984, 259, 10700-10704.
32. Toumi, M. L.; Go, E. P.; Desaire, H. Development of fully functional proteins with novel glycosylation via enzymatic glycan trimming. J. Pharm. Sci. 2009, 98, 2581-2591.
33. Dordal, M. S.; Wang, F. F.; Goldwasser, E. The role of carbohydrate in erythropoietin action. Endocrinology 1985, 116, 2293-2299.
34. Morell, A. G.; Gregoriadis, G.; Scheinberg, I. H.; Hickman, J.; Ashwell, G. The role of sialic acid in determining the survival of glycoproteins in the circulation. J. Biol. Chem. 1971, 246, 1461-1467.
35. Fukuda, M. N.; Sasaki, H.; Lopez, L.; Fukuda, M. Survival of recombinant erythropoietin in the circulation: The role of carbohydrates. Blood 1989, 73, 84-89.
36. Millward, T. A.; Heitzmann, M.; Bill, K.; Langle, U.; Schumacher, P.; Forrer, K. Effect of constant and variable glycosylation on pharmacokinetics of therapeutic antibodies in mice. Biologicals 2008, 36, 41-47.
37. Knibbs, R. N.; Goldstein, I. J.; Ratcliffe, R. M.; Shibuya, N. Characterization of the carbohydrate binding specificity of the leukoagglutinating lectin from Maackia amurensis. J. Biol. Chem. 1991, 266, 83-88.
38. Harle, J.; Bechthold, A. The power of glycosyltransferases to generate bioactive natural compounds. Methods Enzymol. 2009, 458, 309-333.
39. Kozak, M. Initiation of translation in prokaryotes and eukaryotes. Gene. 1999, 234, 187-208.
40. Vermeulen, N. P. E. Prediction of drug metabolism: The case of cytochrome p450 2D6. Current Topics in Medicinal Chemistry. 2003, 3, 1227-1239.
41. Murray, N. E. Type I restriction systems: Sophisticated molecular machines (a legacy of Bertani and Weigle). Microbiology and Molecular Biology Reviews. 2000, 64, 412-434.

We claim:

1. A method comprising:
modifying a protein to include a glycan tag so as to facilitate production of the protein.

2. The method of claim 1 wherein the protein is modified to include more than one glycan tag.

3. The method of claim 1 wherein modifying the protein to include the glycan tag comprises modifying a nucleotide sequence encoding the protein to include a glycosylation unit.

4. The method of claim 3 wherein the glycosylation unit is present proximate to a terminus of the protein.

5. The method of claim 1 wherein modifying the protein to include the glycan tag comprises appending a glycan to the protein as a translational modification during amino acid sequence expression.

6. The method of claim 1 wherein the protein is naturally glycosylated.

7. The method of claim 1 wherein the protein is human growth hormone.

8. The method of claim 1 wherein modifying the protein to include the glycan tag increases the solubility of the protein.

9. The method of claim 1 wherein modifying the protein to include the glycan tag improves the secretion of the protein.

10. A method comprising:
modifying a protein to include at least one modification selected from the group consisting of a glycan tag, a glycosylation unit, and a combination thereof; and then
modifying the protein to at least partially remove at least one modification.

11. The method of claim 10 wherein modifying the protein to include at least one modification comprises modifying a nucleotide sequence encoding the protein to include a glycosylation unit.

12. The method of claim 10 wherein modifying the protein to include at least one modification comprises appending a glycan to the protein as a translational modification during amino acid sequence expression.

13. The method of claim 11 wherein the glycosylation unit is present proximate to a terminus of the protein.

14. The method of claim 11 wherein the glycosylation unit is present at a terminus of the protein.

15. The method of claim 10 wherein modifying the protein to at least partially remove the at least one modification comprises contacting the modification with an enzyme.

16. The method of claim 10 wherein modifying the protein to at least partially remove the at least one modification comprises chemical cleavage.

17. A method comprising:
modifying a protein to include a glycan tag; and
retaining the protein by affinity chromatography.

18. The method of claim 17 wherein modifying the protein to include the glycan tag comprises modifying a nucleotide sequence encoding the protein to include a glycosylation site.

19. The method of claim 17 wherein modifying the protein to include the glycan tag comprises appending the glycan tag to the protein as a translational modification during amino acid sequence expression.

20. A composition comprising:
a nucleic acid sequence encoding a start codon;
a nucleic acid sequence encoding a glycosylation unit; and
a nucleic acid sequence encoding a protein,
wherein the nucleic acid sequence encoding in the glycosylation unit is present in a region outside the nucleic acid sequence encoding the protein.

21. The composition of claim 20 further comprising nucleic acid encoding a fusion protein.

22. The composition of claim 20 further comprising nucleic acid encoding for a restriction site.

23. A composition comprising:
a nucleic acid sequence encoding a start codon;
a nucleic acid sequence encoding a glycosylation unit; and
a nucleic acid sequence encoding a protein,
wherein the nucleic acid sequence encoding the glycosylation unit is present inside the nucleic acid sequence encoding the protein and proximate to a terminus of the protein.
